(19) United States
(12) Patent Application Publication
(10) Pub. No.: US 2011/0306566 A1
(43) Pub. Date: Dec. 15, 2011

(54) TOPICAL SKIN TREATMENT KITS

(75) Inventors: Manny MONTALVO, Bridgewater, NJ (US); Joanne ROBINETT, Bridgewater, NJ (US); David N. WIGLEY, Bridgewater, NJ (US)

(73) Assignee: SANOFI, Paris (FR)

(21) Appl. No.: 13/213,471

(22) Filed: Aug. 19, 2011

Related U.S. Application Data
(63) Continuation of application No. PCT/US2010/040699, filed on Jul. 1, 2010.
(60) Provisional application No. 61/222,556, filed on Jul. 2, 2009.

(30) Foreign Application Priority Data
Dec. 15, 2009 (FR) 0958977

(51) Int. Cl. A61K 31/327; B65D 71/00; A61P 17/00; A61P 31/04; A61K 31/7056; A61K 31/728

(52) U.S. Cl. 514/24; 514/714; 514/54; 206/570

(57) ABSTRACT

This invention relates to benzoyl peroxide products for skin treatment, and more particularly to a kit containing a benzoyl peroxide product and a topical moisturizing product. In one embodiment the kit contains BenzaClin® brand clindamycin-benzoyl peroxide gel and Viscontour® brand hyaluronic acid serum.

### FIELD OF THE INVENTION

This invention relates to benzoyl peroxide products for skin treatment, and more particularly to a kit containing a benzoyl peroxide product and a topical moisturizing product.

### BACKGROUND OF THE INVENTION

Acne is an inflammatory disease which is very common at puberty and which occurs in skin areas where sebaceous glands are largest, most numerous, and most active. In its milder forms, acne is a superficial disorder which is evidenced by slight, spotty irritations, and which can be treated satisfactorily by ordinary skin hygiene. However, pilosebaceous follicles occur and result in the formation of pustules, infected cysts and, in extreme cases, canalizing inflamed and infected sacs, which may become extensive and leave permanent, disfiguring scars.

Zhen et al, J. Drugs Dermatol, Aug. 2007, 6(8): 810-6, indicates that topical therapies effective in treatment of acne, may be associated with local effects, such as irritation and dryness. In comparison, subjects treated with an OTC moisturizer (Nivea brand cream) exhibited skin hydration.

Among the topical anti-acne therapies are preparations which include a peroxide, such as benzoyl peroxide, or a combination of a peroxide and an antibiotic, such as an antibiotic of the lincomycin family, are disclosed in U.S. Pat. Nos. 5,466,028, 5,767,098 and 6,013,637, all of which are incorporated herein by reference. However, benzoyl peroxide has been found to have a natural drying effect.

### SUMMARY OF THE INVENTION

In accordance with the present invention, it has been found that a moisturizer can be useful to overcome the drying effects of benzoyl peroxide preparations. These local adverse effects of benzoyl peroxide may become severe, which may reduce patient adherence or require discontinuation, and thus compromising treatment efficacy. Increasing the hydration of the skin with a moisturizer can effectively reduce the local adverse effects caused by the acne treatment, thereby rendering a more successful treatment outcome.

In accordance with the present invention, there is provided a kit comprising a topical acne medication containing benzoyl peroxide and a separate hydration agent for moisturizing the skin.

In a particular embodiment, the topical acne medication comprises benzoyl peroxide and an antibiotic of the lincomycin family as described in the above-cited U.S. Pat. Nos. 5,466,028, 5,767,098 and 6,013,637.

In a further embodiment, the hydration agent comprises a hyaluronic acid formulation. A particular hyaluronic acid formulation suitable for use in the present invention is sold under the trademark Viscontour® Serum.

### DETAILED DESCRIPTION OF THE INVENTION

The kit of the present invention includes a topical acne medication containing benzoyl peroxide. In a particular embodiment, the acne medication comprising benzoyl peroxide in combination with a topically active antibiotic.

One acne medication suitable for use in the present invention comprises benzoyl peroxide and an antibiotic of the lincomycin family. A particularly suitable lincomycin antibiotic for use in the acne medication is clindamycin.

A topical gel comprising clindamycin (1%) as clindamycin phosphate and benzoyl peroxide (5%) is commercially available, and is sold under the name BenzaClin® topical gel. BenzaClin® topical gel contains clindamycin phosphate, (7(S)-chloro-7-deoxylincomycin-2-phosphate). Clindamycin phosphate is a water soluble ester of the semisynthetic antibiotic produced by a 7(S)-chloro-substitution of the 7(R)-hydroxyl group of the parent antibiotic lincomycin. Chemically, clindamycin phosphate is (C₁₈H₃₄CIN₂O₈PS). Clindamycin phosphate has molecular weight of 504.97 and its chemical name is methyl 7-chloro-6,7,8-trideoxy-6-(1-methyl-trans-4-propyl-L-2-pyrrolidinecarboxamido)-1-thio-L-threo-alpha-D-galacto-octopyranoside 2-(dihydrogen phosphate).

BenzaClin® topical gel also contains benzoyl peroxide, for topical use. Chemically, benzoyl peroxide is (C14H10O4). Benzoyl peroxide has a molecular weight of 242.23. Each gram of BenzaClin® topical gel contains, as dispensed, 10 mg (1%) clindamycin as phosphate and 50 mg (5%) benzoyl peroxide in a base of carbomer, sodium hydroxide, dioctyl sodium sulfosuccinate, and purified water.

Viscontour® serum is a topical hyaluronic acid for skin hydration. It contains water, sodium hyaluronate, sodium chloride, sodium monohydrogenphosphate and sodium dihydrogenphosphate. It is distributed in sterile single-use 0.03 fluid ounce ampoules.

### EXAMPLE

A kit is assembled comprising a jar of BenzaClin® clindamycin phosphate/benzoyl peroxide topical gel and one or more ampoules of Viscontour® hyaluronic acid serum. The clindamycin phosphate/benzoyl peroxide gel is applied to a patient's skin, and then the hyaluronic serum is applied.

We claim:

1. A kit comprising separate containers of a topical medication comprising benzoyl peroxide in a pharmaceutically acceptable carrier and a hydrating agent.

2. The kit of claim 1 wherein the topical medication also contains an antibiotic.

3. The kit of claim 2 wherein the antibiotic is clindamycin or a pharmaceutically acceptable salt or ester thereof.

4. The kit of claim 3 wherein the topical medication comprises about 0.01 to about 10 weight percent antibiotic and about 0.1 to about 30 weight percent benzoyl peroxide.

5. The kit of claim 4 wherein the antibiotic is clindamycin phosphate.

6. The kit of claim 5 wherein the topical medication comprises about 1 weight percent clindamycin phosphate and about 5 weight percent benzoyl peroxide.

7. The kit of claim 1 wherein the hydrating agent comprises a hyaluronic acid in a pharmaceutically acceptable carrier.

8. The kit of claim 6 wherein the hydrating agent comprises a hyaluronic acid in a pharmaceutically acceptable carrier.

9. The kit of claim 7 wherein the hydrating agent comprises water, sodium hyaluronate, sodium chloride, sodium monohydrogenphosphate and sodium dihydrogenphosphate.

10. The kit of claim 8 wherein the hydrating agent comprises water, sodium hyaluronate, sodium chloride, sodium monohydrogenphosphate and sodium dihydrogenphosphate.

11. The kit of claim 9 wherein the hydrating agent is Viscontour® brand hyaluronic acid serum.

12. The kit of claim 10 wherein the hydrating agent is Viscontour® brand hyaluronic acid serum.

13. The kit of claim 12 wherein the topical medication is BenzaClin® brand clindamycin-benzoyl peroxide gel.

14. A method of treating a skin disorder or condition in a patient, the method comprising topically administering a medication comprising benzoyl peroxide in a pharmaceutically acceptable carrier, and applying a separate hydrating agent to the skin.

15. The method of claim 14 wherein the medication is topically administered to the skin first and the hydrating agent is applied to the skin second.

16. The method of claim 14 wherein the medication further comprises an antibiotic.

17. The method of claim 16 wherein said antibiotic is clindamycin or a pharmaceutically acceptable salt or ester thereof.

18. The method of claim 17 wherein the antibiotic is clindamycin phosphate.

19. The method of claim 14 wherein the hydrating agent comprises a hyaluronic acid in a pharmaceutically acceptable carrier.

20. The method of claim 18 wherein the hydrating agent comprises a hyaluronic acid in a pharmaceutically acceptable carrier.

21. The method of claims 14 wherein the skin disorder is acne.

22. The method of claims 20 wherein the skin disorder is acne.
